(19) United States
(12) Patent Application Publication
(10) Pub. No.: US 2026/0078344 A1
(43) Pub. Date: Mar. 19, 2026

(54) COMPOSITIONS FOR PROMOTING ACTIVATION OF BONE MARROW-DERIVED STEM CELLS COMPRISING CCL5

(71) Applicant: PUSAN NATIONAL UNIVERSITY INDUSTRY-UNIVERSITY COOPERATION FOUNDATION, Busan (KR)

(72) Inventors: Dongjun Lee, Yangsan-si (KR); Soo-Yeon Woo, Yangsan-si (KR)

(73) Assignee: PUSAN NATIONAL UNIVERSITY INDUSTRY-UNIVERSITY COOPERATION FOUNDATION, Busan (KR)

(21) Appl. No.: 19/303,355

(22) Filed: Aug. 19, 2025

Related U.S. Application Data
(63) Continuation of application No. PCT/KR2025/008442, filed on Jun. 18, 2025.

(30) Foreign Application Priority Data
Sep. 19, 2024 (KR) 10-2024-0126435

(51) Int. Cl. C12N 5/0789; A61K 35/28; A61P 19/08

(52) U.S. Cl. CPC C12N 5/0647; A61K 35/28; A61P 19/08; C12N 2501/10; C12N 2501/21; C12N 2501/999

(57) ABSTRACT

The present disclosure relates to a composition for promoting the activation of bone marrow-derived stem cells, including C-C motif chemokine ligand 5 (CCL5), wherein, when CCL5 is administered together with growth-related oncogene beta (GROβ) and AMD3100, it not only increases the migration of hematopoietic stem cells from bone marrow to peripheral blood, but also enhances the engraftment efficiency when the hematopoietic stem cells are transplanted into recipients.

### CROSS-REFERENCE TO RELATED APPLICATION

This application is a Continuation of PCT International Patent Application No. PCT/KR2025/008442 filed on Jun. 18, 2025, which claims priority to Korean Patent Application No. 10-2024-0126435 filed on Sep. 19, 2024 which are all hereby incorporated by reference in their entirety.

### BACKGROUND OF THE INVENTION

The present application is supported by the National Research Foundation of Korea under Ministry of Science and ICT, Ministry of Trade, Industry and Energy, Ministry of Health & Welfare, Ministry of Food and Drug Safety for the following research projects:
1) A study on development of new concept erythrocyte differentiation and proliferation restoration technology through identification of differentiation dynamics of highly efficient mobilized hematopoietic stem progenitor cells based on niche enhancing factors (RS-2023-KH140743).
2) A study on research center for molecular control of cancer cell diversity (2022R1A5A2027161).
3) A study on mechanical study on the differentiation control of immature leukemia/cancer stem cells based on key factors and treatment of blood cancer in the bone marrow microenvironment (RS-2024-00333287).

### 1. Field of the Invention

The present disclosure relates to a composition for promoting activation of bone marrow-derived stem cells comprising CCL5.

### 2. Description of the Related Art

Hematopoietic stem cells are the first parent cells of blood cells such as white blood cells, red blood cells, and platelets, and blood cells produced in the bone marrow migrate to peripheral blood after maturation. Most patients with blood disorders have defective hematopoietic stem cells and are unable to produce healthy, functional blood cells with their own hematopoietic stem cells. Therefore, most patients with blood disorders undergo treatment by transplanting healthy hematopoietic stem cells, which is called hematopoietic stem cell transplantation.

Hematopoietic stem cell transplantation is a representative means of treating blood diseases such as various leukemias, aplastic anemia, lymphoma, blood cancers, and multiple myeloma, as well as immunodeficiency and autoimmune diseases. For hematopoietic stem cell transplantation, the recipient is administered large doses of anticancer drugs or receive radiation to the entire body to suppress functions of the patient's problematic bone marrow cells to proceed with the bone marrow transplantation from a healthy donor. For hematopoietic stem cell transplantation, collection of hematopoietic stem cells from a donor is required, and the effectiveness of the treatment depends on the engraftment rate of the donor's hematopoietic stem cells when injected into the recipient.

In most cases, in order to gather hematopoietic stem cells, the collection involves insertion of a needle into the bone behind the diaphysis. In recent years, however, methods of collecting hematopoietic stem cells from peripheral blood through injection of drugs that induce hematopoietic stem cells from the bone marrow to the peripheral blood are developing. A representative example in the use is a method of collecting hematopoietic stem cells using G-CSF, but it requires daily injections for 4 to 5 days, and 150 to 200 ml of blood per 1 kg of the donor's body weight must be processed with a blood component collection device to obtain sufficient hematopoietic stem cells. Therefore, there are limitations in collecting hematopoietic stem cells at high levels in a short period of time. Additionally, it often takes a considerable amount of time consumed for the hematopoietic stem cells to settle in the recipient after transplantation, and the success of the treatment depends on how quickly the bone marrow function recovers.

Therefore, in order to effectively treat diseases that require bone marrow transplantation, methods of easily extracting hematopoietic stem cells from peripheral blood while improving an engraftment rate after transplantation are being continuously studied.

### SUMMARY OF THE INVENTION

### Problem to be Solved by the Invention

An object of the present disclosure is to provide a novel means capable of improving collection and therapeutic effects of bone marrow-derived stem cells that may be utilized for treatment of diseases requiring bone marrow transplantation, by demonstrating that C-C motif chemokine ligand 5 (CCL5), a niche enhancing factor, not only increases migration of bone marrow-derived hematopoietic stem cells into peripheral blood but also increases an engraftment rate of the hematopoietic stem cells migrated into the peripheral blood in a recipient.

### Means for Solving the Problem

The present disclosure provides a composition for promoting activation of bone marrow-derived stem cells, including a C-C motif chemokine ligand 5 (CCL5) protein as an active ingredient.

In addition, the present disclosure provides a method of activating bone marrow-derived stem cells in vitro or ex vivo, comprising contacting the cells with the composition comprising a C-C motif chemokine ligand 5 (CCL5) protein.

In addition, the present disclosure provides a pharmaceutical composition for treating a bone marrow disease, comprising, as an active ingredient, bone marrow-derived stem cells isolated from a donor administered with a C-C motif chemokine ligand 5 (CCL5) protein, growth-related oncogene beta (GROβ), and AMD3100, which is a CXCR4 antagonist.

In addition, the present disclosure provides a method of treating a bone marrow disease in a subject in need thereof, comprising administering to the subject a composition comprising, as an active ingredient, bone marrow-derived stem cells isolated from a donor administered with a C-C motif chemokine ligand 5 (CCL5) protein, growth-related oncogene beta (GROβ), and AMD3100, which is a CXCR4 antagonist.

### Effects of the Invention

According to the present disclosure, when C-C motif chemokine ligand 5 (CCL5), a niche enhancing factor, is injected in combination with growth related oncogene beta (GROβ) and AMD3100, a CXCR4 antagonist, which are known to promote migration of hematopoietic stem cells from bone marrow to peripheral blood, it was demonstrated that not only the migration of hematopoietic stem cells from bone marrow to peripheral blood increased, but also an engraftment rate increased when the hematopoietic stem cells are injected into a recipient receiving bone marrow transplantation, such that it is possible to provide a C-C motif chemokine ligand 5 (CCL5) protein, growth-related oncogene beta (GROβ), and AMD3100, which is a CXCR4 antagonist, as a composition for promoting an activity of bone marrow-derived stem cells, and thus the activated bone marrow-derived stem cells may be provided as a new treatment means for bone marrow diseases that require bone marrow transplantation.

### BRIEF DESCRIPTION OF THE DRAWINGS

FIG. 1 shows results of evaluating effects of fibroblast growth factor 21 (FGF21) and C-C motif chemokine ligand 5 (CCL5), which are niche factors in bone marrow cells, on proliferation of hematopoietic stem cells.

FIG. 2 shows results of evaluating effects of GROβ and AMD3100 on the number of hematopoietic stem cells and progenitor cells in bone marrow cells.

FIG. 3 shows results of evaluating effects of GROβ and AMD3100 on migration of hematopoietic stem cells in mice.

FIG. 4 shows results of evaluating effects of co-administration of GROβ and AMD3100 with a niche enhancing factor on migration of hematopoietic stem cells in mice.

FIG. 5 shows results of evaluating whether an engraftment capacity of activated hematopoietic stem cells changes in recipient mice when GROβ, AMD3100, and CCL5 are co-administered to donor mice.

FIG. 6 shows results of analyzing effects of CCL5 deficiency on the increased migration of hematopoietic stem cells by GROβ and AMD3100.

### DETAILED DESCRIPTION OF THE INVENTION

The terms used in this specification are selected from general terms that are most widely used currently as much as possible while taking into account the functions of the present disclosure, but these may vary depending on the intention of those skilled in the art, precedents, or the emergence of new technologies. Additionally, in certain cases, there are terms arbitrarily selected by the applicant, and in such cases, meanings thereof will be described in detail in the description of the relevant disclosure. Therefore, the terms used herein should not be simply defined as names of terms, but based on the meaning of the terms and the overall content of the present disclosure.

Unless otherwise defined, all terms used herein, including technical or scientific terms, have the same meaning as commonly understood by those of ordinary skill in the art to which the present disclosure pertains. Terms defined in commonly used dictionaries should be interpreted to have a meaning consistent with those in the context of the relevant art and not be interpreted in an idealized or overly formal sense unless expressly defined in this application.

Hereinafter, the present disclosure will be described in more detail.

The present disclosure provides a composition for promoting activation of bone marrow-derived stem cells, including a C-C motif chemokine ligand 5 (CCL5) protein as an active ingredient.

The composition further includes growth-related oncogene beta (GROβ) and AMD3100, which is a CXCR4 antagonist.

The bone marrow-derived stem cells are hematopoietic stem cells and progenitor cells, and specifically, the bone marrow-derived stem cells are any one or more cells selected from among LK (Lin⁻cKit⁺) cells, LKS (Lin⁻cKit⁺Sca1⁺) cells, hematopoietic progenitor cells (HPCs) (Lin⁻cKit⁺Sca1⁺CD48⁺), and hematopoietic stem cells (HSCs) (Lin⁻cKit⁺Sca1⁺CD48⁻CD150⁺).

The activation of the bone marrow-derived stem cells means that the migration of the hematopoietic stem cells existing in the bone marrow into the peripheral blood increases following injection of the composition, indicating that collection of hematopoietic stem cells is facilitated due to the migration to the peripheral blood, with a remarkably excellent engraftment rate in recipients requiring bone marrow transplantation since the collected hematopoietic stem cells are activated.

In addition, the present disclosure provides a pharmaceutical composition for treating a bone marrow disease, comprising, as an active ingredient, bone marrow-derived stem cells isolated from a donor administered with a C-C motif chemokine ligand 5 (CCL5) protein, growth-related oncogene beta (GROβ), and AMD3100, which is a CXCR4 antagonist.

The bone marrow-derived stem cells have remarkably improved engraftment capacity within the recipient.

The bone marrow disease is any one or more diseases selected from the group consisting of acute myeloid leukemia, acute lymphocytic leukemia, chronic myeloid leukemia, chronic lymphocytic leukemia, aplastic anemia, multiple myeloma, malignant lymphoma, myeloproliferative neoplasm, myelodysplastic syndrome, paroxysmal nocturnal hemoglobinuria, immunodeficiency, hemoglobin dystrophy, Fanconi anemia, systemic lupus erythematosus, and induced secondary bone marrow failure.

The pharmaceutical composition of the present disclosure may be manufactured in the form of a unit dose by formulating using a carrier that is pharmaceutically acceptable or manufactured by encapsulating in a large-capacity container, in accordance with a method that may be easily carried out by those with ordinary skill in the art to which the disclosure pertains.

The pharmaceutically acceptable carriers are those commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additive included in the pharmaceutical composition is not particularly limited and may be appropriately adjusted within the content range used in conventional formulations.

The pharmaceutical composition may be formulated in the form of one or more external preparations selected from the group consisting of injectable formulations such as aqueous solutions, suspensions, and emulsions, pills, capsules, granules, tablets, creams, gels, patches, sprays, ointments, plasters, lotions, liniments, pastes, and cataplasmas.

The pharmaceutical composition of the present disclosure may include a pharmaceutically acceptable carrier and diluent that are additionally present for formulation. The pharmaceutically acceptable carriers and diluents include, but are not limited to, excipients such as starch, sugars, and mannitol, fillers and extenders such as calcium phosphate, cellulose derivatives such as carboxymethylcellulose and hydroxypropylcellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be those that are biologically and physiologically friendly to a subject. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oil-based suspensions, powder preparation, granules, emulsions, hard capsules, soft capsules, syrups, and elixirs. When administered parenterally, it may be formulated as injection solutions, suppositories, powder for respiratory inhalation, aerosols for sprays, ointments, powder for application, oils, and creams.

The dosage of the pharmaceutical composition of the present disclosure may vary depending on the patient's condition, weight, age, sex, health status, dietary constitution specificity, nature of the preparation, severity of disease, administration time for composition, method of administration, duration or interval of administration, excretion rate, and drug form, and it may be appropriately selected by a person skilled in the art. For example, it may range from about 0.1 to 10,000 mg/kg but is not limited thereby, and it may be administered once or several times a day.

The pharmaceutical composition may be administered orally or parenterally (e.g., applied intravenously, subcutaneously, intraperitoneally, or topically) depending on the desired method. The pharmaceutically effective amount and effective dosage of the pharmaceutical composition of the present disclosure may vary by the preparation method of the pharmaceutical composition, type of administration, administration time, and administration route, and those with ordinary skill in the art may easily determine and prescribe the effective dosage for the desired treatment. The administration of the pharmaceutical composition of the present disclosure may be conducted once a day or in several divided doses.

In addition, the present disclosure provides a method of treating a bone marrow disease, comprising administering to a subject in need thereof a composition comprising, as an active ingredient, bone marrow-derived stem cells isolated from a donor administered with a C-C motif chemokine ligand 5 (CCL5) protein, growth-related oncogene beta (GROβ), and AMD3100, which is a CXCR4 antagonist.

Since the method utilizes the composition described above, redundant descriptions are omitted to avoid excessive complexity of this specification.

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### Example 1. Effects of FGF21 and CCL5, Which are Niche Enhancing Factors, on Proliferation of Hematopoietic Stem Cells

The effects of fibroblast growth factor 21 (FGF21) and C-C motif chemokine ligand 5 (CCL5), which are factors of the hematopoietic stem cell niche in bone marrow, on proliferation of hematopoietic stem cells were evaluated in mouse bone marrow cells. FGF21 and CCL5 used in the Example were purchased from PeproTech. After separating the femur and tibia of the mouse, both ends of the bones were cut out, and bone marrow was extracted by passing a buffer for flow cytometry into the bones using a syringe. After removing red blood cells in the bone marrow with ACK lysis buffer to obtain lymphocytes, FGF21 or CCL5 were treated at a concentration of 200 ng/mL followed by culture for 48 hours. After 48 hours of culture, lymphocytes were collected and treated with fluorescence-labeled antibodies to be subjected to an analysis on a proportion of LKS cells (Lin⁻cKit⁺Sca1⁺), which are undifferentiated hematopoietic stem cells, and HPC cells (Lin⁻cKit⁺Sca1⁺CD48⁺), which are hematopoietic progenitor cells, via flow cytometry.

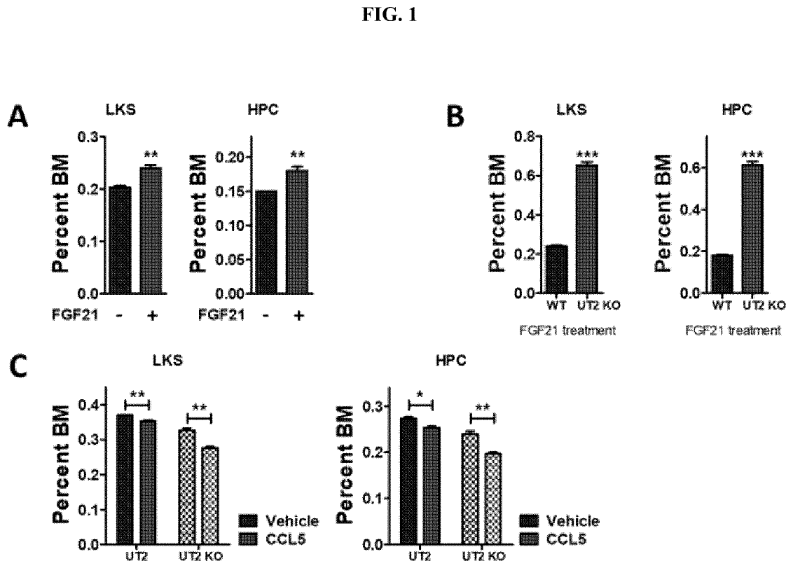

As shown in FIG. 1A, it was found that treatment of mouse bone marrow with FGF21, a niche enhancing factor, resulted in an increase in the number of hematopoietic stem cells and progenitor cells. As shown in FIG. 1B, when FGF21 was treated to the bone marrow of UT2-deficient (UT2 KO) mice (Mx1 Cre), it was found that the number of hematopoietic stem cells and progenitor cells increased more than that of the wild type (WT). As shown in FIG. 1C, treatment of mouse bone marrow with CCL5, a niche enhancing factor, resulted in a decrease in the number of hematopoietic stem cells and progenitor cells, and the number of hematopoietic stem cells and progenitor cells was further decreased in the bone marrow of UT2-deficient (UT2 KO) mice.

From the above results, it was found that FGF21, a niche enhancing factor, increased the number of hematopoietic stem cells and progenitor cells, but CCL5 decreased the number of hematopoietic stem cells and progenitor cells. The above results indicate that FGF21 and CCL5 have different effects on the proliferation capacity of hematopoietic stem cells and progenitor cells, and UT2 deficiency in hematopoietic stem cells and progenitor cells enhances the effects of FGF21 and CCL5.

### Example 2. Effects of GROβ and AMD3100 on Hematopoietic Stem Cells and Progenitor Cells

Growth-related oncogene beta (GROβ), which is a CXCR2 agonist, and AMD3100, which is a CXCR4 antagonist, promote migration of hematopoietic stem cells from the bone marrow to the peripheral blood. The effects of GROβ and AMD3100 on the number of hematopoietic stem cells and progenitor cells in bone marrow were evaluated. GROβ and AMD3100 used in the Example were purchased from PeproTech. After separating the femur and tibia of the mouse, both ends of the bones were cut out, and bone marrow was extracted by passing a buffer for flow cytometry into the bones using a syringe. After removing red blood cells in the bone marrow with ACK lysis buffer to obtain lymphocytes, 5 μL/mL AMD3100 and/or 2.5 μg/mL GROβ were treated followed by culture for 48 hours. After 48 hours of culture, lymphocytes were collected and treated with fluorescence-labeled antibodies to be subjected to an analysis on a proportion of lineage negative cells (Lin−), which are undifferentiated hematopoietic stem cells, LK cells (Lin⁻cKit⁺), LKS cells (Lin⁻cKit⁺Sca1⁺), HPC cells (Lin⁻cKit⁺Sca1⁺CD48⁺), which are hematopoietic stem cells, and HSC cells (Lin⁻cKit⁺Sca1⁺CD48⁻CD150⁺), which are hematopoietic stem cells, via flow cytometry.

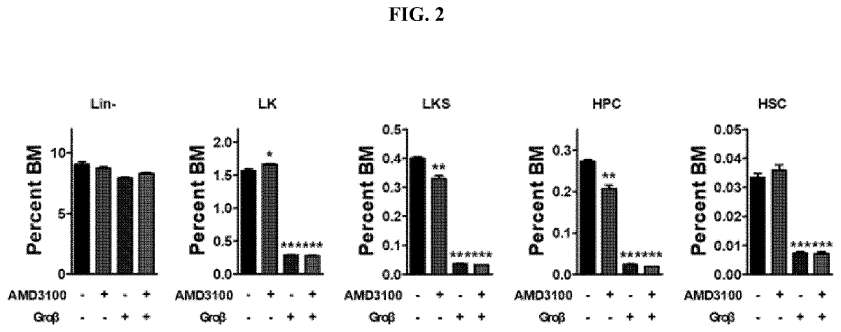

As shown in FIG. 2, treatment of AMD3100 alone to mouse bone marrow decreased the number of LKS cells and HPC cells, and treatment GROβ all decreased the number of LK cells, LKS cells, HPC cells, and HSC cells.

The above results demonstrate that GROβ reduces the number of hematopoietic stem cells and progenitor cells more than AMD3100. The above results show that effects of GROβ and AMD3100 on undifferentiated hematopoietic stem cells, indicating that the effects of GROβ on undifferentiated hematopoietic stem cells are superior to that of AMD3100.

### Example 3. Effects of GROβ and AMD3100 on Migration of Hematopoietic Stem Cells in Mice

To evaluate the effects of GROβ and AMD3100 on hematopoietic stem cell migration, the evaluation was conducted in upstream of mTORC2 (UT2) mice (UT2^{fl/fl}) and mice in which UT2 is deficient in hematopoietic cells (Mx1Cre UT2^{fl/fl}). Mice were injected subcutaneously with 2.5 mg/kg CXCL2 (Groβ) and 5 mg/kg AMD3100 (Plefixafor). After 15 minutes, blood was collected from the mouse and placed in 10 mM EDTA to prevent blood clotting, red blood cells were removed with ACK lysis buffer, and lymphocytes were extracted. After treating lymphocytes with fluorescence-labeled antibodies, the proportion of lineage negative cells (Lin−), which are undifferentiated hematopoietic stem cells, and LKS cells (Lin⁻cKit⁺Sca1⁺) was analyzed via flow cytometry.

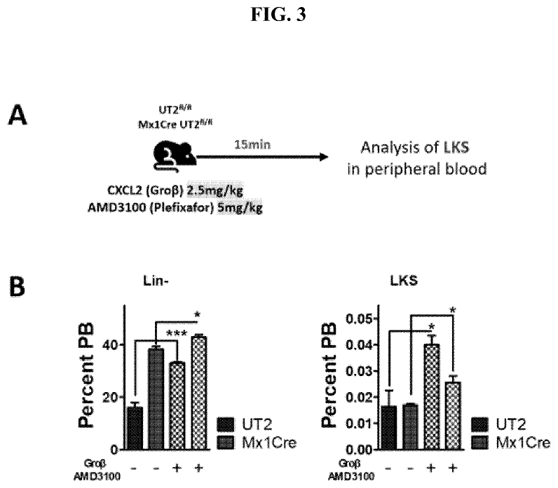

As shown in FIG. 3, the number of undifferentiated bone marrow cells was found to increase in the peripheral blood of mice administered GROβ and AMD3100 compared to an untreated control group. In UT2-deficient mice, LKS cell migration was also increased in mice administered GROβ and AMD3100, but the increase was lower than that of the UT2 mice.

The above results demonstrate that GROβ and AMD3100 have the effect of inducing the migration of hematopoietic stem cells in the bone marrow to the peripheral blood, and in the case of UT2 deficiency in hematopoietic stem cells and progenitor cells, the effect of GROβ and AMD3100 on the migration of hematopoietic stem cells was reduced. The above results showed the stem cell migration ability of GROβ and AMD3100, indicating that UT2 is involved in the stem cell migration ability of GROβ and AMD3100.

### Example 4. Effects of Co-Administration of GROβ and AMD3100 With Niche Enhancing Factors on Hematopoietic Stem Cell Migration in Mice

To evaluate the effect of co-administration of GROβ and AMD3100 with niche enhancing factors on hematopoietic stem cell migration, evaluation was conducted in UT2 mice (UT2^{fl/fl}). Mice were injected subcutaneously with 2.5 mg/kg CXCL2 (Groβ), 5 mg/kg AMD3100 (Plefixafor), 1 mg/kg CCL5, and 1 mg/kg FGF21. After 15 minutes, blood was collected from the mouse and placed in 10 mM EDTA to prevent blood clotting, red blood cells were removed with ACK lysis buffer, and lymphocytes were extracted. After treating lymphocytes with fluorescence-labeled antibodies, the proportion of LKS cells (Lin⁻cKit⁺Sca1⁺), which are a population of undifferentiated hematopoietic stem cells, was analyzed via flow cytometry.

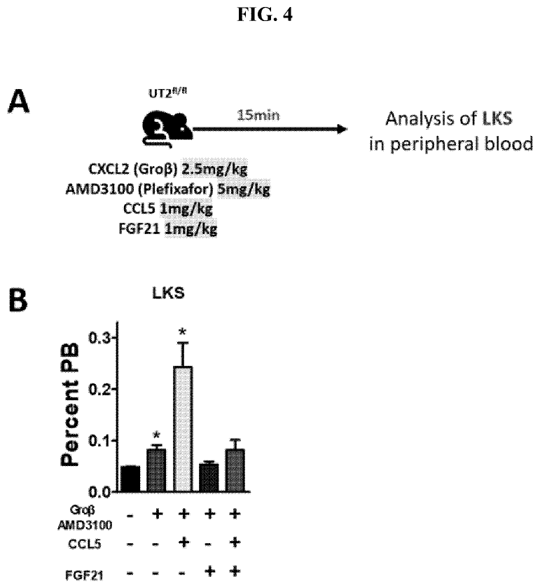

As shown in FIG. 4, the number of undifferentiated bone marrow cells was found to increase in the peripheral blood of mice administered GROβ and AMD3100 compared to the untreated control group. When CCL5 was administered together with GROβ and AMD3100, the number of LKS cells in peripheral blood was shown to be increased by more than two times. Through the above results, it was found that the co-administration of GROβ and AMD3100 along with CCL5 had the effect of activating hematopoietic stem cells and significantly increasing their migration.

### Example 5. Evaluation on Effects of Co-Administration of GROβ and AMD3100 With CCL5 on an Engraftment Capacity of Donor Hematopoietic Stem Cells

To evaluate the engraftment capacity of hematopoietic stem cells migrated to peripheral blood by co-administration of GROβ, AMD3100, and CCL5, donor mice were co-administered with GROβ, AMD3100, and CCL5, and then hematopoietic stem cells were collected from the peripheral blood and administered to recipient mice, so as to analyze whether donor-derived hematopoietic stem cells are engrafted. Boy/J CD45.1 mice were used as donor mice, and 2.5 mg/kg CXCL2 (Groβ), 5 mg/kg AMD3100 (Plefixafor), and 1 mg/kg CCL5 were injected subcutaneously into the donor mice. After 15 minutes, blood was collected from the mouse and placed in 10 mM EDTA to prevent blood clotting, then red blood cells were removed with ACK lysis buffer, and lymphocytes were extracted. To evaluate the engraftment capacity of donor-derived hematopoietic stem cells, the extracted lymphocytes were mixed in a 2:1 ratio with lymphocytes extracted from the bone marrow of C57BL/6 CD45.2 mice having similar genotype for competitive transplantation. C57BL/6 CD45.2 mice, which have a similar genotype to the donor mice, were used as recipient mice. The mixed lymphocytes were transplanted by injection into the tail vein of recipient mice that received busulfan to remove bone marrow cells. One month after transplantation, blood was collected from the recipient mice and placed in 10 mM EDTA to prevent blood clotting, red blood cells were removed with ACK lysis buffer, and lymphocytes were extracted. After treating lymphocytes with fluorescence-labeled antibodies, the engraftment rate of cells derived from donor mice was determined by flow cytometry.

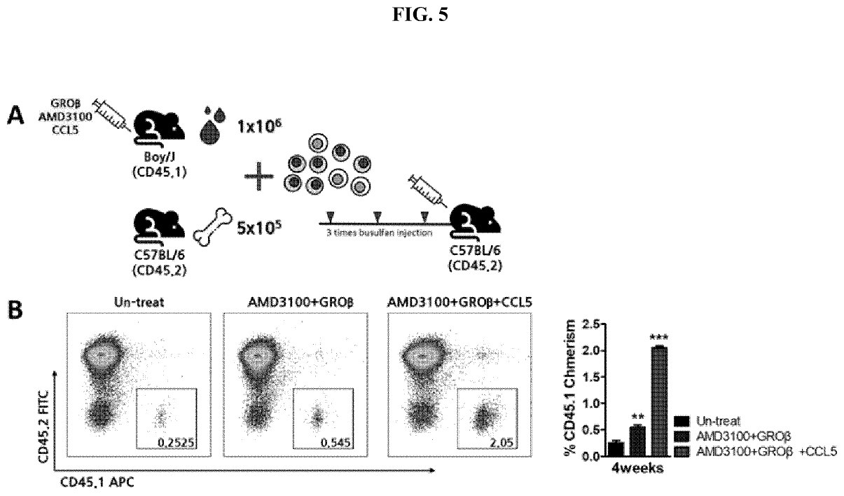

As shown in FIG. 5, as a result of identifying the proportion of cells derived from CD45.1 donor mice in the blood of recipient mice 4 weeks after bone marrow transplantation, lymphocytes extracted from donors co-administered with GROβ, AMD3100, and CCL5 were found at a higher proportion in the blood of recipient mice compared to a case when only GROβ and AMD3100 were administered. The above results demonstrate that bone marrow collected by co-administration of GROβ, AMD3100, and CCL5 to bone marrow donor mice exhibits the higher engraftment capacity in recipient mice.

### Example 6. Effects of CCL5 Deficiency on Migration of Hematopoietic Stem Cells Increased by GROβ and AMD3100

WT mice and CCL5 KO mice with CCL5 deficiency were injected subcutaneously with 2.5 mg/kg CXCL2 (Groβ) and 5 mg/kg AMD3100 (Plefixafor). After 15 minutes, blood was collected from the mouse and placed in 10 mM EDTA to prevent blood clotting, red blood cells were removed with ACK lysis buffer, and lymphocytes were extracted. After treating lymphocytes with fluorescence-labeled antibodies, the proportion of LKS cells (Lin⁻cKit⁺Sca1⁺), which are an undifferentiated hematopoietic stem cell population, and HPC cells (Lin⁻cKit⁺Sca1⁺CD48⁺), which are hematopoietic progenitor cells, was analyzed via flow cytometry.

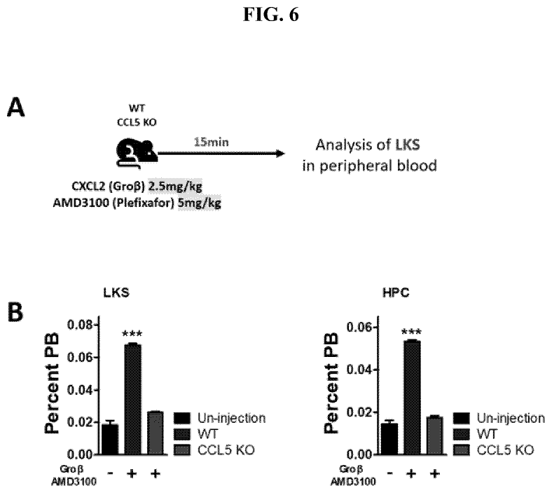

As shown in FIG. 6, the number of LKS cells and HPC cells in the peripheral blood of WT mice administered with GROβ and AMD3100 increased compared to that of untreated control WT mice, showing that the migration of LKS cells and HPC cells to the peripheral blood increased. However, in CCL5-deficient mice, it was found that administration of GROβ and AMD3100 reduced the increased migration of LKS cells and HPC cells into peripheral blood. The above results demonstrate that CCL5 deficiency reduces GROβ and AMD3100-induced migration of hematopoietic stem cells to the peripheral blood.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

The numerical range includes the numerical values defined in the above range. Any maximum numerical limit given throughout this specification includes any lower numerical limit as if the lower numerical limit were explicitly written out. Any minimum numerical limit given throughout this specification includes any higher numerical limit as if that higher numerical limit were explicitly written out. Any numerical limit given throughout this specification will include any better numerical range within the broader numerical range, as if the narrower numerical limit were explicitly written.

What is claimed is:

1. A method of activating bone marrow-derived stem cells in vitro or ex vivo, comprising contacting the bone marrow-derived stem cells with a composition comprising a C-C motif chemokine ligand 5 (CCL5) protein.

2. The method of claim 1, wherein the composition further comprises growth-related oncogene beta (GROβ) and AMD3100, which is a CXCR4 antagonist.

3. The method of claim 1, wherein the bone marrow-derived stem cells are hematopoietic stem cells and progenitor cells.

4. The method of claim 1, wherein the bone marrow-derived stem cells are any one or more cells selected from among LK (Lin−cKit+) cells, LKS (Lin−cKit+Sca1+) cells, hematopoietic progenitor cells (HPCs) (Lin−cKit+Sca1+CD48+), and hematopoietic stem cells (HSCs) (Lin−cKit+Sca1+CD48−CD150+).

5. The method of claim 1, wherein the activation of the bone marrow-derived stem cells leads to an increase in migration of stem cells from the bone marrow to peripheral blood.

6. A method of treating a bone marrow disease in a subject in need thereof, comprising:
administering to the subject a therapeutically effective amount of a pharmaceutical composition comprising, as an active ingredient, bone marrow-derived stem cells isolated from a donor administered with a C-C motif chemokine ligand 5 (CCL5) protein, growth-related oncogene beta (GROβ), and AMD3100, which is a CXCR4 antagonist.

7. The method of claim 6, wherein the bone marrow-derived stem cell has improved engraftment capacity within a recipient.

8. The method of claim 6, wherein the bone marrow disease is any one or more diseases selected from the group consisting of acute myeloid leukemia, acute lymphocytic leukemia, chronic myeloid leukemia, chronic lymphocytic leukemia, aplastic anemia, multiple myeloma, malignant lymphoma, myeloproliferative neoplasm, myelodysplastic syndrome, paroxysmal nocturnal hemoglobinuria, immunodeficiency, hemoglobin dystrophy, Fanconi anemia, systemic lupus erythematosus, and induced secondary bone marrow failure.

9. The method of claim 6, wherein the bone marrow-derived stem cells are hematopoietic stem cells and progenitor cells.

10. The method of claim 6, wherein the bone marrow-derived stem cells are any one or more cells selected from among LK (Lin⁻cKit⁺) cells, LKS (Lin⁻cKit⁺Sca1⁺) cells, hematopoietic progenitor cells (HPCs) (Lin⁻cKit⁺Sca1⁺CD48⁺), and hematopoietic stem cells (HSCs) (Lin⁻cKit⁺Sca1⁺CD48⁻CD150⁺).
